SEQUENCE LISTING (2 sequences)

SEQ ID NO: 1 - protein, 7 aa, Artificial Sequence
  BASED ON RATTUS
TLVVHEK

SEQ ID NO: 2 - protein, 12 aa, Artificial Sequence
  BASED ON HOMO SAPIENS
HVGDLGNVTADK

(19) United States
(12) Patent Application Publication
(10) Pub. No.: US 2014/0302520 A1
(43) Pub. Date: Oct. 9, 2014

(54) METABOLISM OF SOD1 IN CSF

(71) Applicant: The Washington University, St. Louis, MO (US)

(72) Inventors: Randall J. Bateman, St. Louis, MO (US); Timothy M. Miller, St. Louis, MO (US); Ling Munsell, St. Louis, MO (US)

(21) Appl. No.: 14/352,560

(22) PCT Filed: Oct. 17, 2012
(86) PCT No.: PCT/US2012/060597

§ 371 Date: Apr. 17, 2014

Related U.S. Application Data
(60) Provisional application No. 61/547,890, filed on Oct. 17, 2011.

(51) Int. Cl. G01N 33/577

(52) U.S. Cl. CPC G01N 33/577
USPC 435/7.1

(57) ABSTRACT

The disclosure relates to methods for the diagnosis and treatment of neurological and neurodegenerative diseases, disorders, and associated processes. Specifically, the disclosure relates to a method for measuring the metabolism of central nervous system derived biomolecules in a subject in vivo. Further disclosed are methods for measuring the in vivo metabolism (e.g. the rate of synthesis, the rate of clearance) of neurally derived biomolecules, such as superoxide dismutase 1 (SOD1).

### ACKNOWLEDGEMENT OF FEDERAL RESEARCH SUPPORT

The present invention was made, at least in part, with funding from the National Institutes of Health, grant no. T35 DK074375. Accordingly, the United States Government may have certain rights in this invention.

### FIELD OF THE INVENTION

The invention relates to methods for the diagnosis and treatment of neurological and neurodegenerative diseases, disorders, and associated processes. The invention also relates to a method for measuring the metabolism of central nervous system derived biomolecules in a subject in vivo.

### BACKGROUND OF THE INVENTION

Amyotrophic lateral sclerosis (ALS) is a neurodegenerative disease marked by the progressive loss of motor neurons in the spinal cord and brain resulting in weakness, atrophy of skeletal muscles, loss of motor function, paralysis, and eventual death from respiratory failure 3-5 years post diagnosis. While 90% of ALS cases are sporadic, about 10% are dominantly-inherited. Of these familial cases, approximately 20% are due to dominant mutations in the enzyme Cu,Zn-superoxide dismutase 1 (SOD1), a homodimeric metalloenzyme that catalyzes the conversion of superoxide anion to hydrogen peroxide and molecular oxygen. Over 150 mutations have been characterized for this 153 amino acid protein that affect many aspects of its structure and function, such as catalytic activity, Cu and Zn binding sites, dimerization, intramolecular disulfide bond formation, and folding. As such, multiple hypotheses have been proposed to explain the mechanism behind mutant SOD1 toxicity, yet not one has been definitively proven. How this ubiquitously expressed protein imparts a selective toxicity to motor neurons of the spinal cord and primary motor cortex remains unknown.

Many neurodegenerative diseases are the result of the accumulation of mutant proteins. Although the expression of some of these proteins is largely limited to the CNS, others like SOD1 are ubiquitously expressed in all tissues of the body. Despite this universal expression, SOD1 mutations result in selective death of motor neurons. One attractive hypothesis is that the CNS handles misfolded, mutant proteins less effectively than other non-neuronal tissues. Indeed, global proteomics approaches using stable isotope labeling kinetics have shown that brain proteins have the lowest turnover rate, even if identical proteins or protein complexes are compared between tissues. These studies suggest that less efficient protein turnover in the CNS may set the stage for misfolded SOD1 accumulation that allows for pathology development. However, the comparison of turnovers rate of wild-type and mutant SOD1 between non-neuronal and neuronal tissues has never been studied and may yield valuable information regarding the tissue specificity of the disease.

A need exists, therefore, for a sensitive, accurate, and reproducible method for measuring the in vivo metabolism of biomolecules in the CNS. In particular, a method is needed for measuring the in vivo fractional synthesis rate and clearance rate of proteins associated with a neurodegenerative disease, e.g., the metabolism of SOD1 in ALS.

### SUMMARY OF THE INVENTION

One aspect of the invention provides methods for measuring the in vivo metabolism (e.g. the rate of synthesis, the rate of clearance) of neurally derived biomolecules, such as SOD1.

An additional aspect of the invention encompasses kits for measuring the in vivo metabolism of neurally derived proteins in a subject, whereby the metabolism of the protein may be used as a predictor of a neurological or neurodegenerative disease, a monitor of the progression of the disease, or an indicator of the effectiveness of a treatment for the disease.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

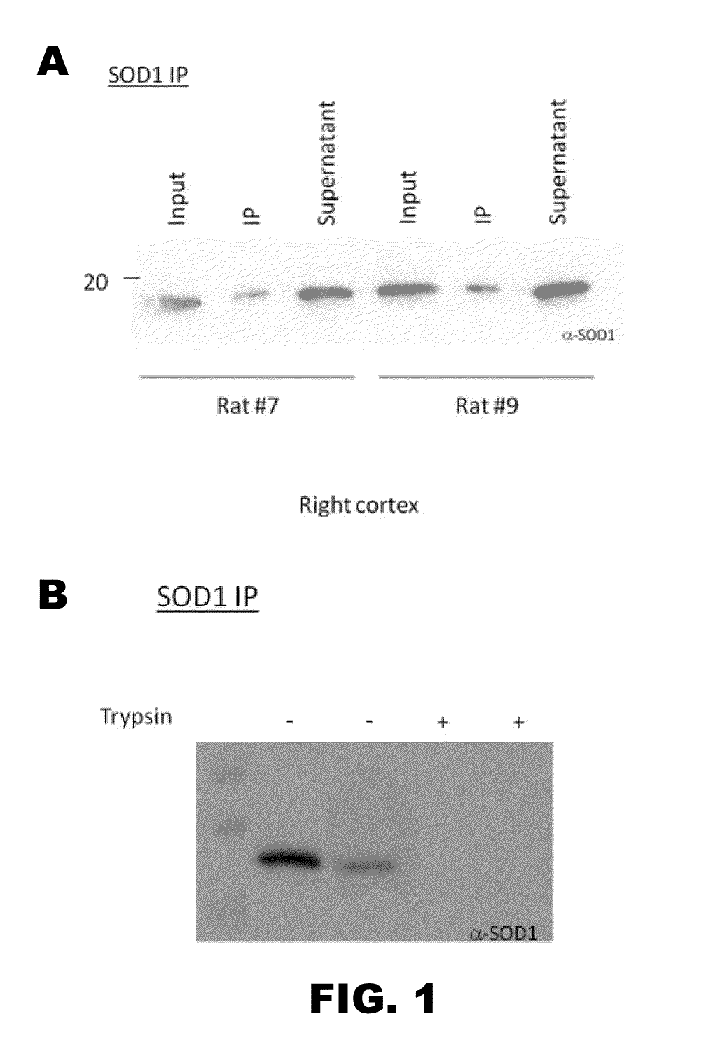

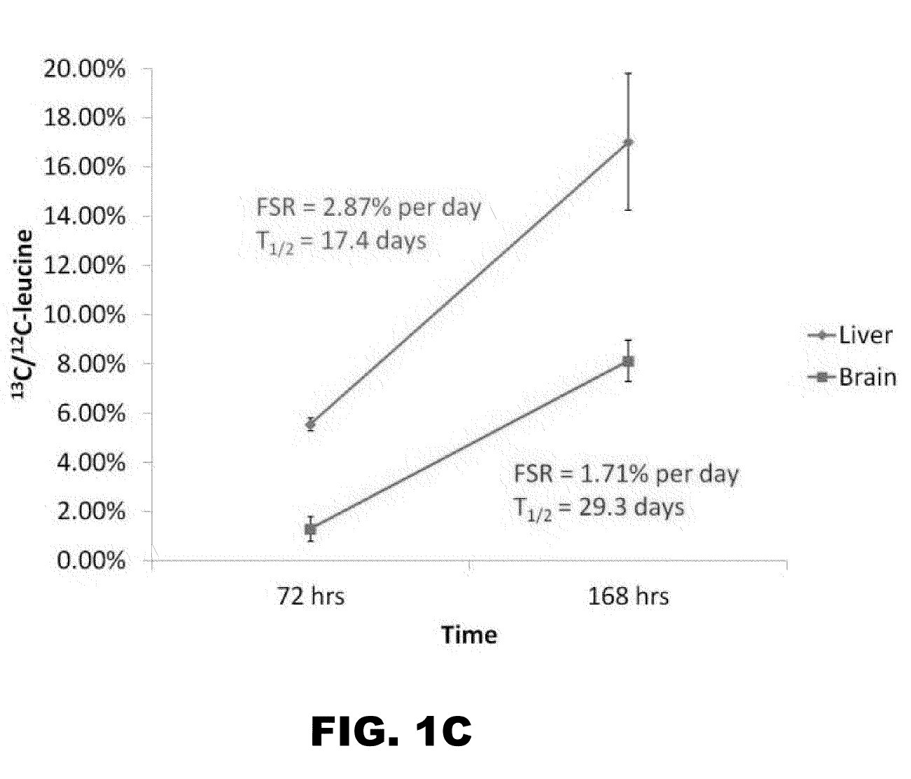

FIG. 1 depicts images and graphs showing successful labeling of SOD1 WT (SOD1^{WT})in cortex from transgenic rats. A) Western blot showing immunoprecipitation with anti-SOD1 antibody from SOD1 WT rat cortex. B) Formic acid elution and trypsin digestion of SOD1 from rat cortex. C) Time-dependent incorporation of ¹³C-leucine in the SOD1 tryptic fragment TLWHEK in brain and liver. FSR=fractional synthesis rate; T_{1/2}=SOD1 WT half life.

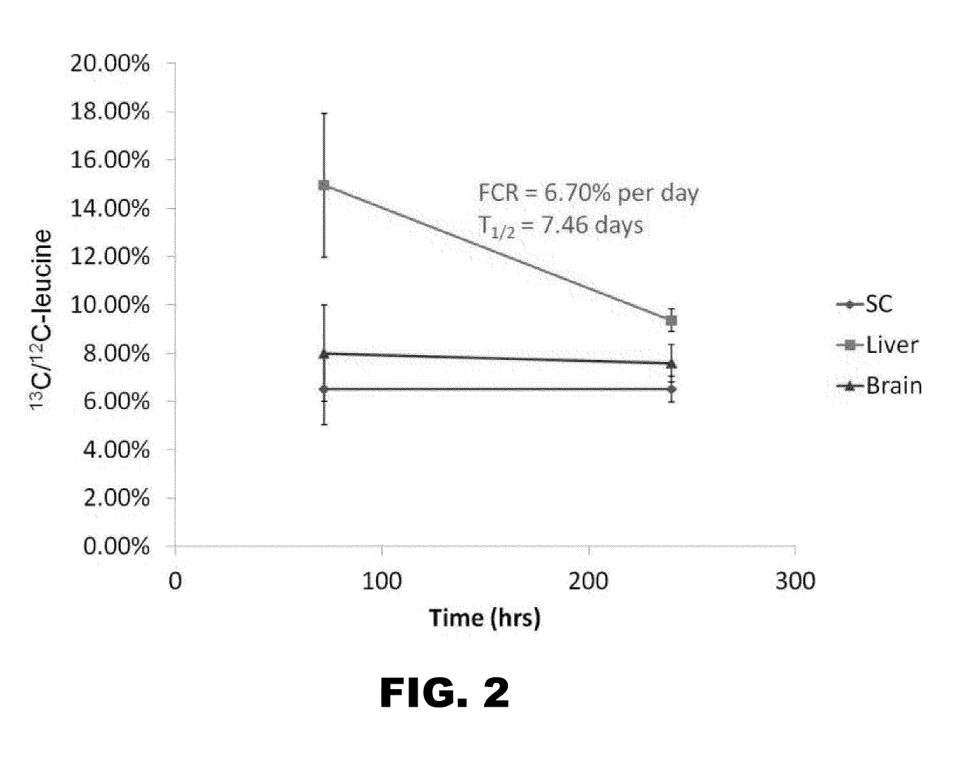

FIG. 2 depicts a graph showing tissue-specific differences in SOD1 G39A (SOD1^{G39A}) turnover. FCR=fractional clearance rate; T_{1/2}=SOD1 G39A half life; SC=spinal cord.

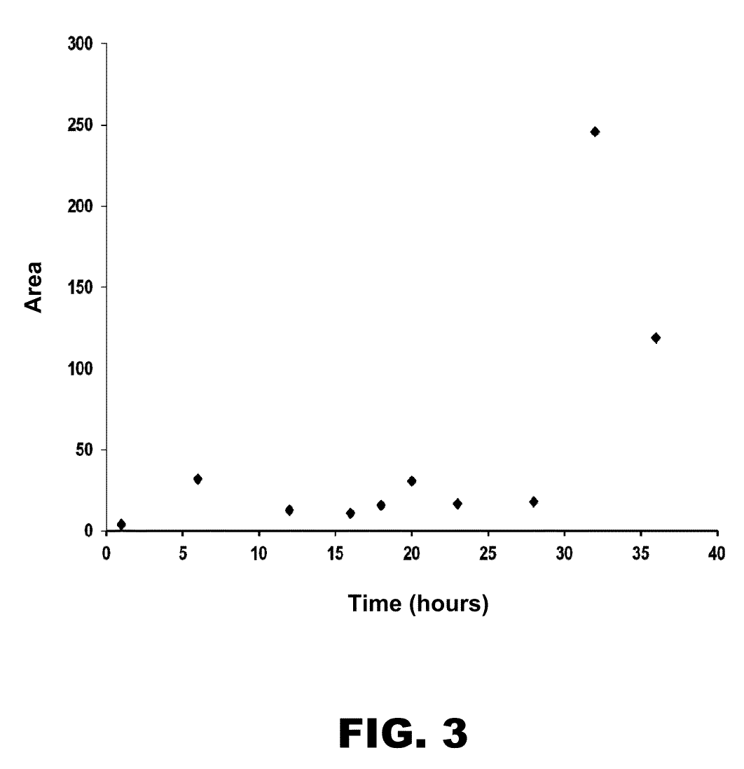

FIG. 3 depicts a graph showing mass spectrometry data of SOD1 from ¹³C₆-leucine labeled human CSF. Graphed on the Y-axis is the Area; graphed on the X-axis is time (hours). The graph shows that labeled human CSF samples showed a continued increase in labeled to unlabeled SOD1 ratio even at the latest time points.

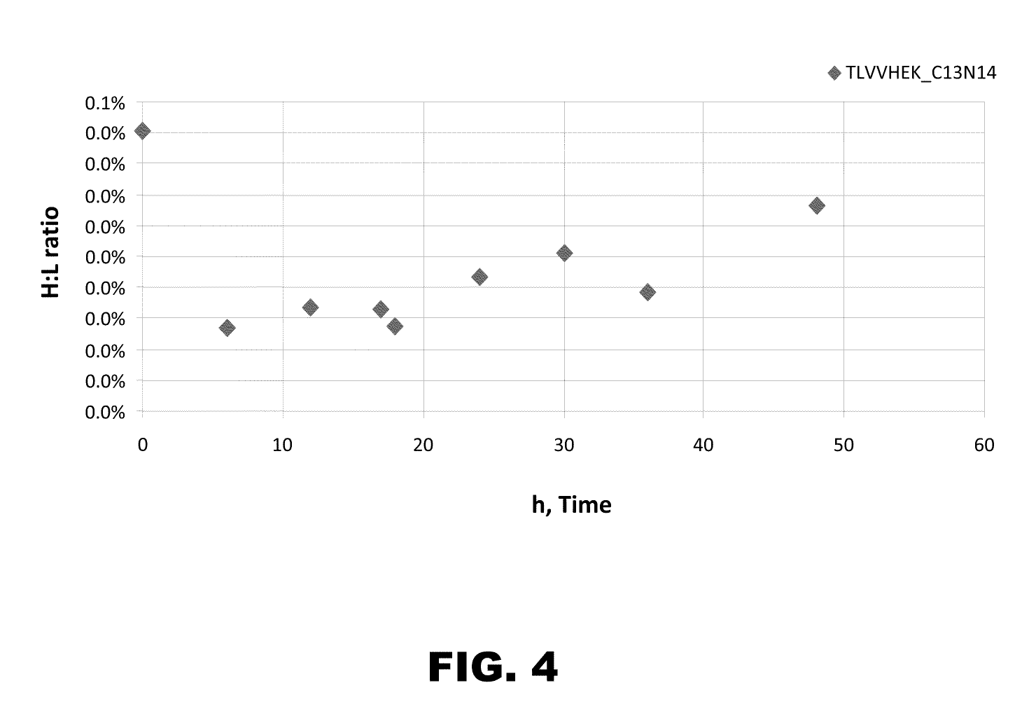

FIG. 4 depicts a graph showing mass spectrometry data plotting H:L ratio along the Y-axis and time (hours) along the X-axis. Samples are human CSF samples and the squares represent the trypsin peptide fragment TLVVHEK_C13N14 (SEQ ID NO:1).

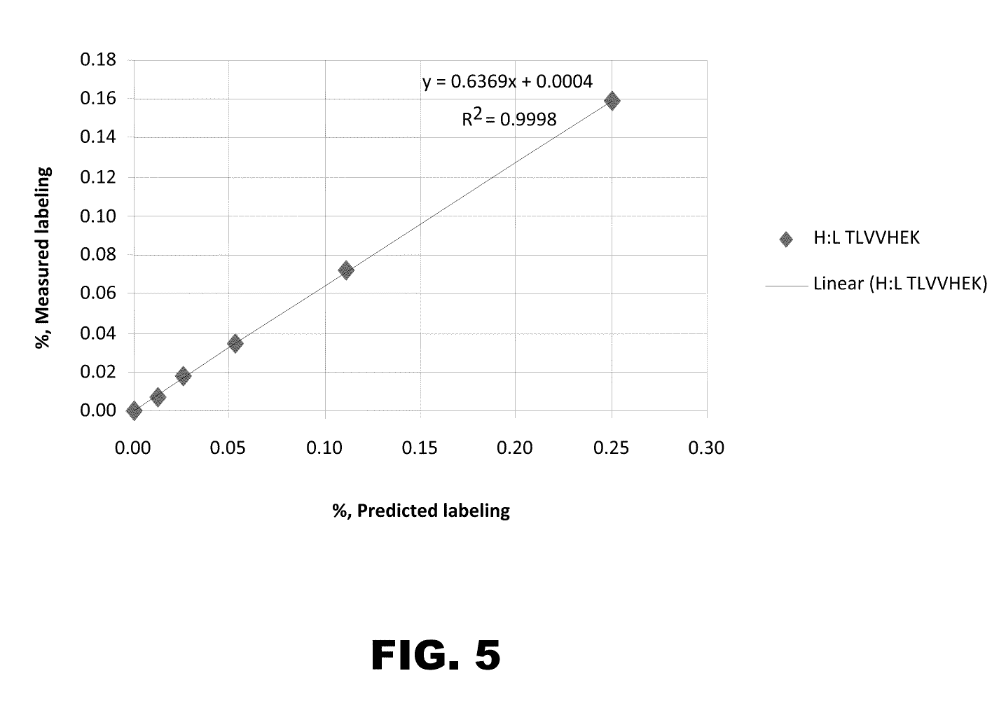

FIG. 5 depicts a graph showing a calibration curve of TLVVHEK_C13N14(SEQ ID NO:1). The percent labeled versus the predicted value is shown with a linear regression line. Note the good linear fit, in addition to the low deviation.

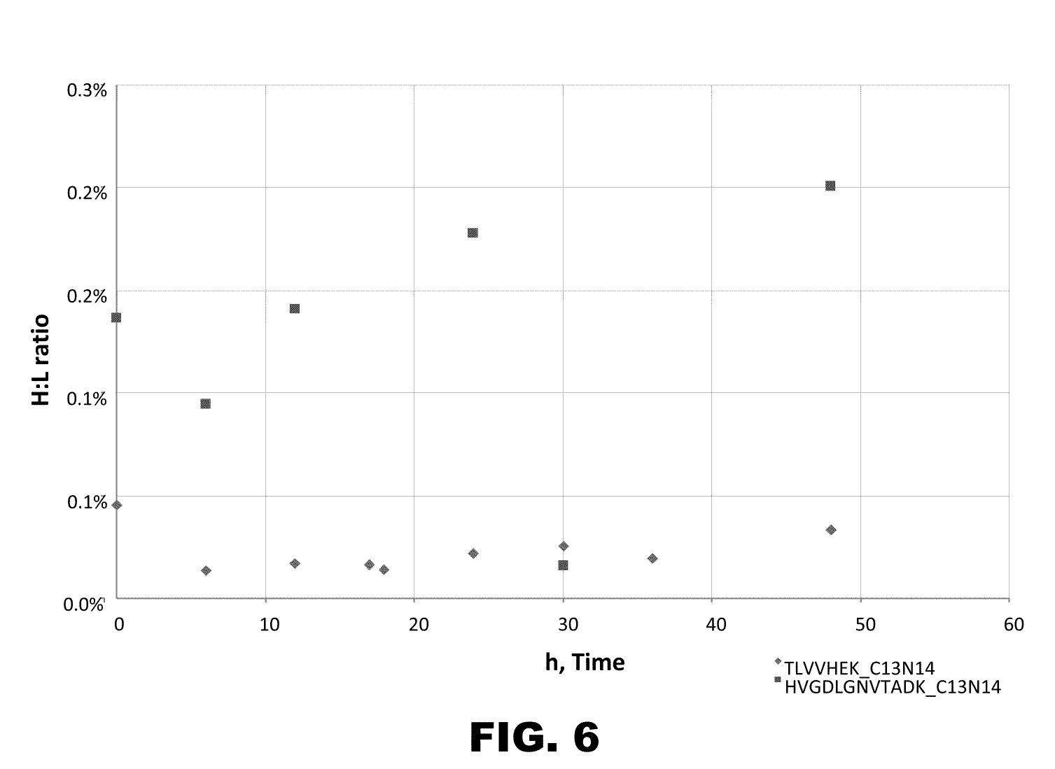

FIG. 6 depicts a graph showing mass spectrometry data plotting H:L ratio along the Y-axis and time (hours) along the X-axis. Samples are human CSF samples. Red squares represent the trypsin peptide fragment HVGDLGNVTADK_C13N14 (SEQ ID NO:2) and blue squares represent TLVVHEK_C13N14 (SEQ ID NO:1).

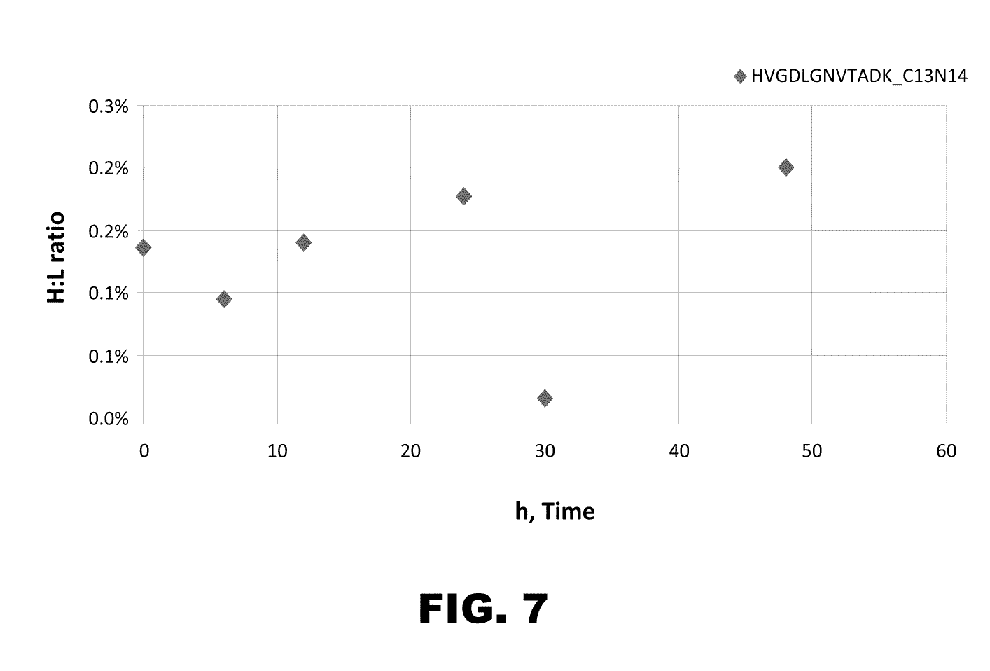

FIG. 7 depicts a graph showing mass spectrometry data plotting H:L ratio along the Y-axis and time (hours) along the X-axis. Samples are human CSF samples and the squares represent the trypsin peptide fragment HVGDLGNVTADK_C13N14 (SEQ ID NO:2).

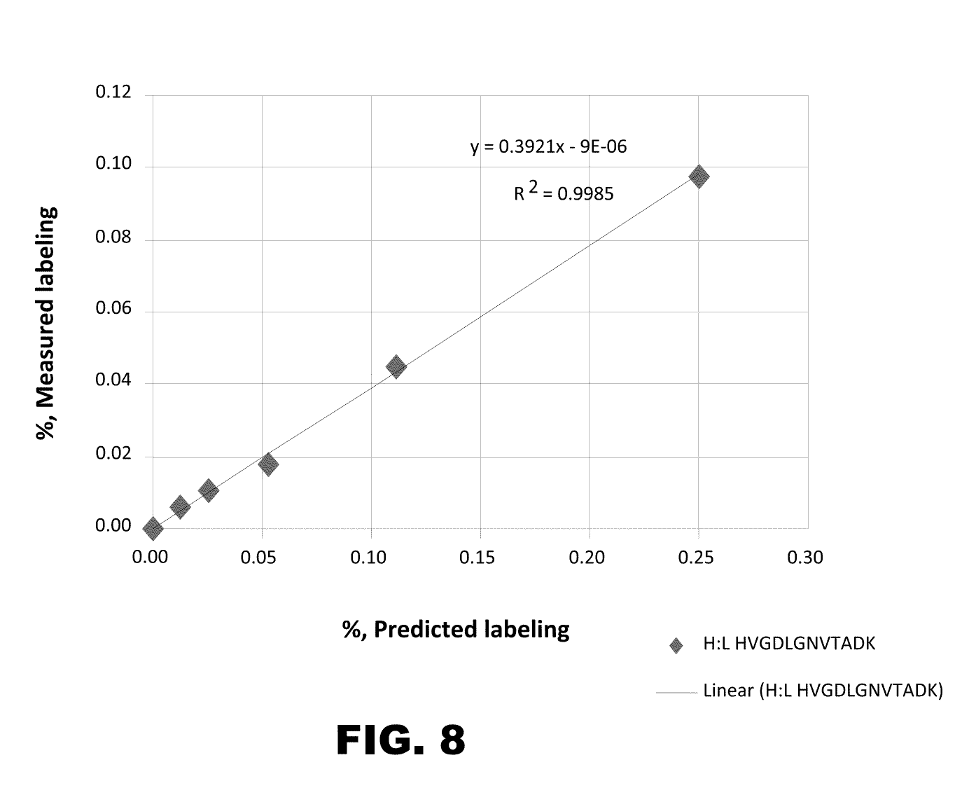

FIG. 8 depicts a graph showing a calibration curve of HVGDLGNVTADK_C13N14 (SEQ ID NO:2). The percent labeled versus the predicted value is shown with a linear regression line. Note the good linear fit, in addition to the low deviation.

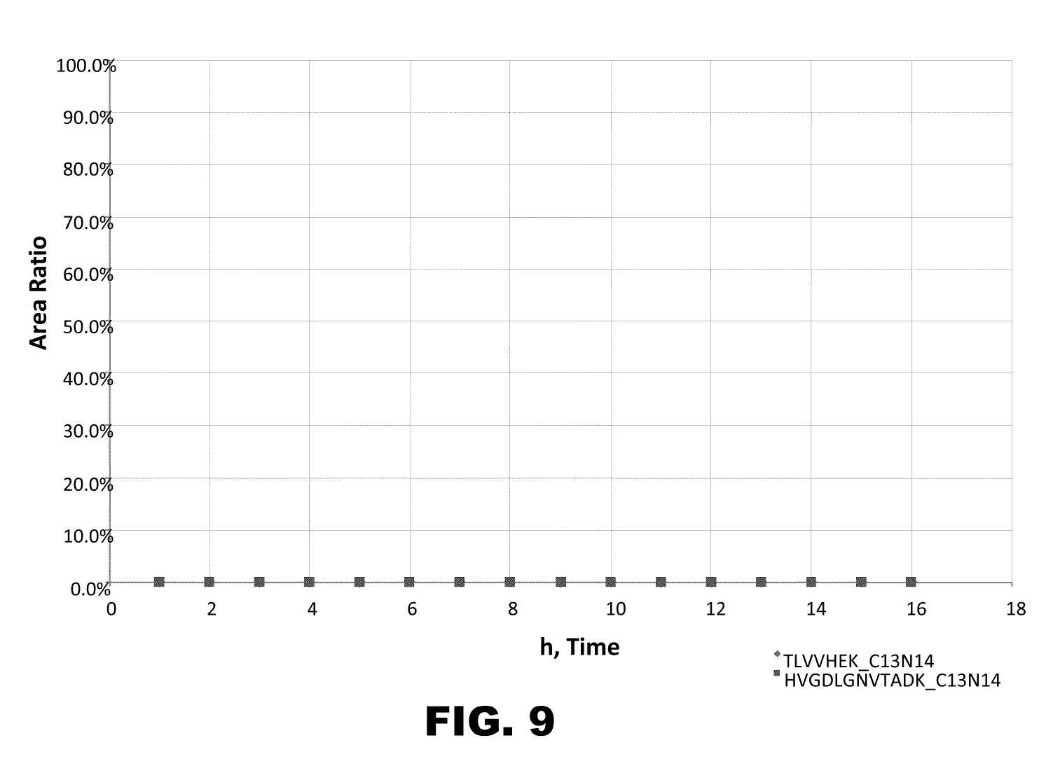

FIG. 9 depicts a graph showing mass spectrometry data plotting Area ratio along the Y-axis and time (hours) along the X-axis. Samples are human CSF samples. Red squares represent the trypsin peptide fragment HVGDLGNVTADK_C13N14 (SEQ ID NO:2) and blue squares represent TLVVHEK_C13N14 (SEQ ID NO:1). Note; the squares overlap completely.

### DETAILED DESCRIPTION OF THE PREFERRED EMBODIMENTS

The present invention relates to determining the synthesis and clearance rates of SOD1. It also provides a method to assess whether a treatment is affecting the production or clearance rate of SOD1 in the CNS relevant to neurological and neurodegenerative diseases. The usefulness of this invention will be evident to those of skill in the art in that one may determine if a treatment alters the synthesis or clearance rate of SOD1. Ultimately, this method may provide a predictive test for the advent of neurological and neurodegenerative diseases, provide a method for more accurate diagnosis, and a means to monitor the progression of such diseases.

### I. Methods for Monitoring the In Vivo Metabolism of Neurally Derived Biomolecules

The current invention provides methods for measuring the in vivo metabolism of SOD1. By using this method, one skilled in the art may be able to study possible changes in the metabolism (synthesis and clearance) of SOD1 in a particular disease state. In addition, the invention permits the measurement of the pharmacodynamic effects of disease-modifying therapeutics in a subject.

In particular, this invention provides a method to label SOD1 as it is synthesized in the central nervous system in vivo; to collect a biological sample containing labeled and unlabeled SOD1; and a means to measure the labeling of SOD1 over time. These measurements may be used to calculate metabolic parameters, such as the synthesis and clearance rates within the CNS, as well as others.

### (a) Degenerative Diseases

Mutations in the gene for superoxide dismutase 1 (SOD1) account for 20% of dominantly inherited amyotrophic lateral sclerosis (ALS) cases. These genetic mutations ultimately produce proteins with toxic properties. Currently, no treatment is available for familial ALS, but it is thought that inhibiting SOD1 production may limit the production of toxic proteins and disease progression. Some treatments focus on lowering SOD1 protein levels in the brain and spinal cord. Decreased SOD1 in brain and spinal cord is reflected in decreased SOD1 in the cerebral spinal fluid (CSF). Thus, CSF SOD1 may be used as a biomarker for the treatment's effectiveness in reducing SOD1 synthesis.

Those of skill in the art will appreciate that, while ALS is the exemplary disease that may be diagnosed or monitored by the invention, the invention is not limited to ALS. It is envisioned that the method of the invention may be used in the diagnosis and assessment of treatment efficacy of several neurological and neurodegenerative diseases, disorders, or processes related to SOD1 metabolism. It is also envisioned that the method of the invention may be used to study the normal physiology, metabolism, and function of the CNS.

It is envisioned that the in vivo metabolism of SOD1 will be measured in a human subject, and in certain embodiments, in a human subject with risk of developing ALS or in a human subject that has been diagnosed with ALS. Alternatively, the in vivo metabolism of biomolecules may be measured in other mammalian subjects. In another embodiment, the subject is a companion animal such as a dog or cat. In another alternative embodiment, the subject is a livestock animal such as a cow, pig, horse, sheep or goat. In yet another alternative embodiment, the subject is a zoo animal. In another embodiment, the subject is a research animal such as a non-human primate or a rodent.

### (b) Labeled Moiety

Several different moieties may be used to label SOD1. Generally speaking, the two types of labeling moieties typically utilized in the method of the invention are radioactive isotopes and non-radioactive (stable) isotopes. In a preferred embodiment, non-radioactive isotopes may be used and measured by mass spectrometry. Preferred stable isotopes include deuterium ²H, ¹³C, ¹⁵N, ^{17 or 18}O, ^{33, 34, or 36}S, but it is recognized that a number of other stable isotope that change the mass of an atom by more or less neutrons than is seen in the prevalent native form would also be effective. A suitable label generally will change the mass of SOD1 under study such that it can be detected in a mass spectrometer. In one embodiment, the labeled moiety is an amino acid comprising a non-radioactive isotope (e.g., ¹³C). In another embodiment, the biomolecule to be measured is a nucleic acid, and the labeled moiety is a nucleoside triphosphate comprising a non-radioactive isotope (e.g., ¹⁵N). Alternatively, a radioactive isotope may be used, and the labeled biomolecules may be measured with a scintillation counter rather than a mass spectrometer. One or more labeled moieties may be used simultaneously or in sequence.

In a preferred embodiment, when the method is employed to measure the metabolism of a protein, the labeled moiety typically will be an amino acid. Those of skill in the art will appreciate that several amino acids may be used to provide the label of SOD1. Generally, the choice of amino acid is based on a variety of factors such as: (1) The amino acid generally is present in at least one residue of the protein or peptide of interest. (2) The amino acid is generally able to quickly reach the site of protein synthesis and rapidly equilibrate across the blood-brain barrier. Leucine is a preferred amino acid to label proteins that are synthesized in the CNS, as demonstrated in the Examples. (3) The amino acid ideally may be an essential amino acid (not produced by the body), so that a higher percent of labeling may be achieved. Non-essential amino acids may also be used; however, measurements will likely be less accurate. (4) The amino acid label generally does not influence the metabolism of the protein of interest (e.g., very large doses of leucine may affect muscle metabolism). And (5) availability of the desired amino acid (i.e., some amino acids are much more expensive or harder to manufacture than others). In one embodiment, ¹³C₆-phenylalanine, which contains six ¹³C atoms, is used to label a neurally derived protein. In a preferred embodiment, 13C₆-leucine is used to label a neurally derived protein. In an exemplary embodiment, ¹³C₆-leucine is used to label SOD1.

There are numerous commercial sources of labeled amino acids, both non-radioactive isotopes and radioactive isotopes. Generally, the labeled amino acids may be produced either biologically or synthetically. Biologically produced amino acids may be obtained from an organism (e.g., kelp/seaweed) grown in an enriched mixture of ¹³C, ¹⁵N, or another isotope that is incorporated into amino acids as the organism produces proteins. The amino acids are then separated and purified. Alternatively, amino acids may be made with known synthetic chemical processes.

### (c) Administration of the Labeled Moiety

The labeled moiety may be administered to a subject by several methods. Suitable methods of administration include intravenously, intra-arterially, subcutaneously, intraperitoneally, intramuscularly, or orally. In a preferred embodiment, the labeled moiety is a labeled amino acid, and the labeled amino acid is administered by intravenous infusion. In another embodiment, labeled amino acids may be orally ingested.

The labeled moiety may be administered slowly over a period of time or as a large single dose depending upon the type of analysis chosen (e.g., steady state or bolus/chase). To achieve steady-state levels of the labeled biomolecule, the labeling time generally should be of sufficient duration so that the labeled biomolecule may be reliably quantified. In one embodiment, the labeled moiety is labeled leucine and the labeled leucine is administered intravenously for at least nine hours. In another embodiment, the labeled leucine is administered intravenously for at least 12 hours. In some embodiments, the labeled leucine is administered intravenously for at least nine, ten, eleven, or twelve hours. In other embodiments, the labeled leucine is administered intravenously for greater than twelve hours.

Those of skill in the art will appreciate that the amount (or dose) of the labeled moiety can and will vary. Generally, the amount is dependent on (and estimated by) the following factors. (1) The type of analysis desired. For example, to achieve a steady state of about 15% labeled leucine in plasma requires about 2 mg/kg/hr over 9 hr after an initial bolus of 2 mg/kg over 10 min. In contrast, if no steady state is required, a large bolus of labeled leucine (e.g., 1 or 5 grams of labeled leucine) may be given initially. (2) The protein under analysis. For example, if the protein is being produced rapidly, then less labeling time may be needed and less label may be needed—perhaps as little as 0.5 mg/kg over 1 hour. However, most proteins have half-lives of hours to days (or weeks) and, so more likely, a continuous infusion for at least 4, 9 or 12 hours may be used at 0.5 mg/kg to 4 mg/kg. And (3) the sensitivity of detection of the label. For example, as the sensitivity of label detection increases, the amount of label that is needed may decrease.

Those of skill in the art will appreciate that more than one label may be used in a single subject. This would allow multiple labeling of the same biomolecule and may provide information on the production or clearance of that biomolecule at different times. For example, a first label may be given to subject over an initial time period, followed by a pharmacologic agent (drug), and then a second label may be administered. In general, analysis of the samples obtained from this subject would provide a measurement of metabolism before AND after drug administration, directly measuring the pharmacodynamic effect of the drug in the same subject.

Alternatively, multiple labels may be used at the same time to increase labeling of the biomolecule, as well as obtain labeling of a broader range of biomolecules.

### (d) Biological Sample

The method of the invention provides that a biological sample be obtained from a subject so that the in vivo metabolism of the labeled SOD1 may be determined. Suitable biological samples include, but are not limited to, bodily fluids or tissues in which SOD1 may be detected. For instance, in one embodiment, the bodily fluid is cerebral spinal fluid (CSF). In another embodiment, the biological sample is a tissue sample. For each type of biological sample, one of skill in the art should recognize that the half-life of SOD1 in the tissue should be determined in order to select sample collection times.

Cerebrospinal fluid may be obtained by lumbar puncture with or without an indwelling CSF catheter. Other types of samples may be collected by direct collection using standard good manufacturing practice (GMP) methods.

In general when the biomolecule under study is a protein, the invention provides that a first biological sample be taken from the subject prior to administration of the label to provide a baseline for the subject. After administration of the labeled amino acid or protein, one or more samples generally would be taken from the subject. As will be appreciated by those of skill in the art, the number of samples and when they would be taken generally will depend upon a number of factors such as: the type of analysis, type of administration, the protein of interest, the rate of metabolism, the type of detection, etc. Different tissues and different mutations in SOD1 may require different collection times.

In one embodiment, the biomolecule is SOD1 and samples of CSF are taken over the course of several days. As the half-life of SOD1 in CSF is generally thought to be greater than 3 weeks, CSF samples may be taken over the course of more than three, four, five, six, seven, eight, nine, or ten days. In some embodiments, one or more samples may be collected once a week for at least 2, 3, 4, 5, 6, 7, 8, or 9 weeks. In other embodiments, samples may be collected on at least one time point prior to the half-life of SOD1 in CSF, and at least one time point greater than the half-life of SOD1. In a particular embodiment, at least one sample may be collected between about 20 days and about 35 days. In another embodiment, at least one sample may be collected between about 20 days and about 30 days. In yet another embodiment, at least one sample may be collected between about 25 days and about 35 days. In this context, ‘about’ means±1 day.

In other embodiments, the biomolecule is SOD1 and samples of liver tissue are taken over the course of several days. For instance, one or more samples may be taken at about 14, 15, 16, 17, 18, 19, or 20 days. In another embodiment, the biomolecule is SOD1 and samples of brain tissue are taken over the course of several days. For instance, one or more samples may be taken at about 25, 26, 27, 28, 29, 30, 31 or 32 days.

Generally speaking, at least two samples should be collected. In some embodiments, two, three, or four samples may be collected. In certain embodiments, more than four samples may be collected.

### (e) Detection

The present invention provides that detection of the amount of labeled SOD1 and the amount of unlabeled SOD1 in the biological samples may be used to determine the ratio of labeled biomolecule to unlabeled SOD1. Generally, the ratio of labeled to unlabeled SOD1 is directly proportional to the metabolism of SOD1. Suitable methods for the detection of labeled and unlabeled SOD1 can and will vary according to the form of SOD1 under study and the type of labeled moiety used to label it. If the biomolecule of interest is a protein and the labeled moiety is a non-radioactively labeled amino acid, then the method of detection typically should be sensitive enough to detect changes in mass of the labeled protein with respect to the unlabeled protein. In a preferred embodiment, mass spectrometry is used to detect differences in mass between the labeled and unlabeled SOD1. In one embodiment, gas chromatography mass spectrometry is used. In an alternate embodiment, MALDI-TOF mass spectrometry is used. In a preferred embodiment, high-resolution tandem mass spectrometry is used.

Additional techniques may be utilized to separate the protein of interest from other proteins and biomolecules in the biological sample. As an example, immunoprecipitation may be used to isolate and purify the protein of interest before it is analyzed by mass spectrometry. Alternatively, mass spectrometers having chromatography setups may be used to isolate proteins without immunoprecipitation, and then the protein of interest may be measured directly. In an exemplary embodiment, the protein of interest is immunoprecipitated and then analyzed by a liquid chromatography system interfaced with a tandem MS unit equipped with an electrospray ionization source (LC-ESI-tandem MS).

The invention also provides that multiple proteins or peptides in the same biological sample may be measured simultaneously. That is, both the amount of unlabeled and labeled protein (and/or peptide) may be detected and measured separately or at the same time for multiple proteins. As such, the invention provides a useful method for screening changes in synthesis and clearance of proteins on a large scale (i.e. proteomics/metabolomics) and provides a sensitive means to detect and measure proteins involved in the underlying pathophysiology. Alternatively, the invention also provides a means to measure multiple types of biomolecules. In this context, for example, a protein and a carbohydrate may be measured simultaneously or sequentially.

### (f) Metabolism Analysis

Once the amount of labeled and unlabeled SOD1 has been detected in a biological sample, the ratio or percent of labeled biomolecule may be determined. If the biomolecule of interest is a protein and the amount of labeled and unlabeled SOD1 has been measured in a biological sample, then the ratio of labeled to unlabeled protein may be calculated. Protein metabolism (synthesis rate, clearance rate, lag time, half-life, etc.) may be calculated from the ratio of labeled to unlabeled protein over time. There are many suitable ways to calculate these parameters. The invention allows measurement of the labeled and unlabeled protein (or peptide) at the same time, so that the ratio of labeled to unlabeled protein, as well as other calculations, may be made. Those of skill in the art will be familiar with the first order kinetic models of labeling that may be used with the method of the invention. For example, the fractional synthesis rate (FSR) may be calculated. The FSR equals the initial rate of increase of labeled to unlabeled protein divided by the precursor enrichment. Likewise, the fractional clearance rate (FCR) may be calculated. In addition, other parameters, such as lag time and isotopic tracer steady state, may be determined and used as measurements of the protein's metabolism and physiology. Also, modeling may be performed on the data to fit multiple compartment models to estimate transfer between compartments. Of course, the type of mathematical modeling chosen will depend on the individual protein synthetic and clearance parameters (e.g., one-pool, multiple pools, steady state, non-steady-state, compartmental modeling, etc.).

The invention provides that the synthesis of protein is typically based upon the rate of increase of the labeled/unlabeled protein ratio over time (i.e., the slope, the exponential fit curve, or a compartmental model fit defines the rate of protein synthesis). For these calculations, a minimum of one sample is typically required (one could estimate the baseline label), two are preferred, and multiple samples are more preferred to calculate an accurate curve of the uptake of the label into the protein (i.e., the synthesis rate).

Conversely, after the administration of labeled amino acid is terminated, the rate of decrease of the ratio of labeled to unlabeled protein typically reflects the clearance rate of that protein. For these calculations, a minimum of one sample is typically required (one could estimate the baseline label), two are preferred, and multiple samples are more preferred to calculate an accurate curve of the decrease of the label from the protein over time (i.e., the clearance rate). The amount of labeled protein in a biological sample at a given time reflects the synthesis rate (i.e., production) or the clearance rate (i.e., removal or destruction) and is usually expressed as percent per hour or the mass/time (e.g., mg/hr) of the protein in the subject.

In an exemplary embodiment, as illustrated in the examples, the in vivo metabolism of SOD1 is measured by administering labeled leucine to a subject over 9 hours and collecting at least one biological samples at a time point greater than 4 days after administration of the label. The biological sample may be collected from CSF. The amount of labeled and unlabeled SOD1 in the biological samples is typically determined by immunopreciptitation followed by LC-ESI-tandem MS. From these measurements, the ratio of labeled to unlabeled SOD1 may be determined, and this ratio permits the determination of metabolism parameters, such as rate of synthesis and rate of clearance of SOD1.

### II. Kits for Diagnosing or Monitoring the Progression or Treatment of Neurological and Neurodegenerative Diseases

The current invention provides kits for measuring SOD1 or monitoring the progression or treatment of a neurological or neurodegenerative disease associated with SOD1 by measuring the in vivo metabolism of a central nervous system-derived protein in a subject. Generally, a kit comprises a labeled amino acid, means for administering the labeled amino acid, means for collecting biological samples over time, and instructions for detecting and determining the ratio of labeled to unlabeled SOD1 so that a metabolic index may be calculated. The metabolic index then may be compared to a metabolic index of a normal, healthy individual or compared to a metabolic index from the same subject generated at an earlier time. In a preferred embodiment, the kit comprises ¹³C₆-leucine or ¹³C₆-phenylalanine, the protein to be labeled is SOD1, and the disease to be assessed is ALS.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Clearance rate” refers to the rate at which the biomolecule of interest is removed.

“Fractional clearance rate” or FCR is calculated as the natural log of the ratio of labeled biomolecule over a specified period of time.

“Fractional synthesis rate” or FSR is calculated as the slope of the increasing ratio of labeled biomolecule over a specified period of time divided by the predicted steady state value of the labeled precursor.

“Isotope” refers to all forms of a given element whose nuclei have the same atomic number but have different mass numbers because they contain different numbers of neutrons. By way of a non-limiting example, ¹²C and ¹³C are both stable isotopes of carbon.

“Lag time” generally refers to the delay of time from when the biomolecule is first labeled until the labeled biomolecule is detected.

“Metabolism” refers to any combination of the synthesis, transport, breakdown, modification, or clearance rate of a biomolecule.

“Metabolic index” refers to a measurement comprising the fractional synthesis rate (FSR) and the fractional clearance rate (FCR) of the biomolecule of interest. Comparison of metabolic indices from normal and diseased individuals may aid in the diagnosis or monitoring of neurological or neurodegenerative diseases.

“Neurally derived cells” includes all cells within the blood-brain-barrier including neurons, astrocytes, microglia, choroid plexus cells, ependymal cells, other glial cells, etc.

“Steady state” refers to a state during which there is insignificant change in the measured parameter over a specified period of time.

“Synthesis rate” refers to the rate at which the biomolecule of interest is synthesized.

In metabolic tracer studies, a “stable isotope” is a nonradioactive isotope that is less abundant than the most abundant naturally occurring isotope.

“Subject” as used herein means a living organism having a central nervous system. In particular, the subject is a mammal. Suitable subjects include research animals, companion animals, farm animals, and zoo animals. The preferred subject is a human.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Stable Isotope Labeling Kinetics in a Rat Model of ALS

Transgenic rats overexpressing human SOD1 WT were fed a leucine-free chow for two weeks prior to labeling with ¹³C-leucine in order to acclimate the rats to the novel diet. During this acclimation period, normal ¹²C-leucine (100 mg/day) was provided in the drinking water, sweetened slightly with sucrose. After the acclimation period, the drinking water was replaced with ¹³C-leucine (100 mg/day) in order to label the animals. Data presented in FIGS. 1A and 1B show that a 7 day labeling period results in sufficient SOD1 label in brain and liver. After the 7 day labeling period, the animals were chased with normal ¹²C-leucine, after which animals were sacrificed at specific time points, perfused with PBS/heparin, and brain, spinal cord, liver, and kidney was harvested, flash frozen in liquid nitrogen, and stored at −80° C.

SOD1 was immunoprecipitated from tissue lysates using an anti-SOD1 mouse monoclonal antibody (Sigma) covalently linked to magnetic Dynabeads (Invitrogen). Briefly, tissues are thawed on ice and mechanically homogenized using a hand blender in NP-40 lysis buffer (1% NP-40, 150 mM Tris, protease inhibitors). SOD1 is immunoprecipitated from 500 μg of total protein using 50 μL of anti-SOD1 crosslinked beads overnight. The beads are washed three times in PBS and SOD1 eluted from the beads with 50 μL of formic acid. The formic acid eluent is lyophilized via vacuum and resuspended in 25 mM NaHCO₃ buffer. 400 ng of sequencing grade trypsin is added to the samples and digestion allowed to proceed at 37° C. for 18 hours. Samples are again lyophilized via vacuum and resuspended in 20 μL of 0.05% formic acid in preparation for the LC/MS run (Xevo). The ratio of labeled to unlabeled SOD1 is determined by comparing the area under the curve for the peptide TLWHEK (SEQ ID NO:1) with or without ¹³C-leucine, respectively. From these labeling data, the fractional synthesis rate (FSR) and fractional clearance rate (FCR) can be calculated, depending on if the tissues were taken during the labeling or chase period, respectively.

### Example 2

### Tissue-Specific FSR for SOD1 WT Transgenic Rats

As can be seen in FIG. 1C, SOD1 from two different tissues (brain and liver) was immunoprecipitated, digested, and analyzed from transgenic rats overexpressing human SOD1 WT. It is clear from the data that a tissue-specific difference exists between the brain and liver. Specifically, brain synthesizes SOD1 at a rate that is 60% of liver. As FSR and FCR are intrinsically linked to maintain protein steady-state levels, the half-life of SOD1 WT in the brain and liver can be estimated to be 29.3 and 17.4 days, respectively. The long half-life for brain is in agreement with a prior study looking at SOD1-YFP half-life in the spinal cords of transgenic mice.

### Example 3

### Tissue-Specific FCR for SOD1 G93A Transgenic Rats

Transgenic rats overexpressing human SOD1 G93A were fed a leucine-free chow for two weeks prior to labeling with ¹³C-leucine in order to acclimate the rats to the novel diet. During this acclimation period, normal ¹²C-leucine (100 mg/day) was provided in the drinking water, sweetened slightly with sucrose. After the acclimation period, the drinking water was replaced with ¹³C-leucine (100 mg/day) in order to label the animals. After a 7 day labeling period, the animals were chased with normal ¹²C-leucine, after which animals were sacrificed at 3-days and 10-days post-label, perfused with PBS/heparin, and brain, spinal cord, liver, and kidney was harvested, flash frozen in liquid nitrogen, and stored at −80° C.

As shown in FIG. 2, the FCR of G93A in liver is determined to be 6.70% per day, which translates into a half-life of 7.46 days. Brain and spinal cord were not significantly different enough between time points to have confidence in calculating an FCR, but the data show that these tissues degrade SOD1G93A much slower than in liver. This suggests that the spacing between collection points, for certain CSF embodiments, should be greater than 3 days.

### Example 4

### Stable Isotope Labeling of SOD1 in Humans

CSF samples were previously obtained from healthy volunteers after administration of a stable isotope-labeled amino acid (¹³C₆-leucine). Briefly, participants had 2 IVs and one lumbar catheter placed. In one IV, ¹³C₆-labeled leucine was infused for 9 or 12 hours. Each hour, plasma and CSF were obtained through the other IV and the lumbar catheter, respectively. Samples were taken at 1-hour time intervals for 36 hours. Further details can be found in Batemen et. al. Nat. Med. 2006, which is incorporated by reference herein.

Monoclonal anti-superoxide dismutase antibody was purified using a Protein A IgG Purification Kit (Pierce) and coupled to magnetic beads using a M-270 Epoxy Dynabeads Antibody Coupling Kit (Invitrogen). SOD1 was immunoprecipitated from CSF samples using the anti-SOD1 antibody coupled to the magnetic beads. The immunoprecipitated SOD1 samples were then digested with trypsin for 18 hours at 37° C. and run on LC/MS.

After trypsin digestion, two SOD1 peptides, TLWHEK (SEQ ID NO:1) and HVGDLGNVTADK (SEQ ID NO:2), showed the highest intensity from mass spectrometry and were used for subsequent studies and analysis. Mass spectrometry of ¹³C-leucine labeled human CSF samples at time points, 0 hrs, 6 hrs, 12 hrs, 17 hrs, 18 hrs, 24 hrs, 30 hrs, and 36 hrs was performed.

Previously, this stable isotype labeling method was performed on an extracellular protein, amyloid beta. Because SOD1 is an intracellular protein, it is likely that the rate of SOD1 excretion into the CSF is much slower than anticipated or that the half-life of SOD1 is very long (many days to weeks). As shown in FIG. 3, it was not possible to detect a peak in the strength of the mass spec signal for the peptides of interest within the 36 hour time point.

1. A method for measuring the in vivo metabolism of SOD1 in a subject, the SOD1 being synthesized in the central nervous system, the method comprising:
(a) administering a labeled moiety to the subject, the labeled moiety being capable of crossing the blood brain barrier and incorporating into SOD1 as the SOD1 is synthesized in the central nervous system of the subject;
(b) obtaining at least one biological sample from the subject, the biological sample comprising a SOD1 fraction labeled with the moiety and a SOD1 fraction not labeled with the moiety; and
(c) detecting the amount of labeled SOD1 and the amount of unlabeled SOD1, wherein the ratio of labeled SOD1 to unlabeled SOD1 is directly proportional to the metabolism of SOD1 in the subject.

2. The method of claim 1, wherein the labeled moiety is an atom, or a molecule with a labeled atom.

3. The method of claim 2, wherein the atom is a radioactive isotope.

4. The method of claim 2, wherein the atom is a non-radioactive isotope.

5. The method of claim 4, wherein the non-radioactive isotope is selected from the group consisting of ²H, ¹³C, ¹⁵N, ¹⁷O, ¹⁸O, ³³S, ³⁴S, and ³⁶S.

6. The method of claim 1, wherein the labeled moiety is administered to the subject intravenously.

7. The method of claim 1, wherein the biological sample is cerebral spinal fluid.

8. The method of claim 1, further comprising separating the labeled SOD1 fraction and the unlabeled SOD1 fraction from the biological sample.

9. The method of claim 8, wherein the protein is separated by immunoprecipitation.

10. The method of claim 8, wherein the amount of labeled SOD1 and the amount of unlabeled SOD1 is detected by mass spectrometry.

11. The method of claim 1, wherein the subject is a rodent.

12. The method of claim 11, wherein the mammal is a human.

13. A kit for diagnosing or monitoring the progression or treatment of a neurological or neurodegenerative disease in a subject, the kit comprising:
(a) a labeled amino acid;
(b) means for administering the labeled amino acid to the subject, whereby the labeled amino acid is capable of crossing the blood brain barrier and incorporating into and labeling SOD1 as SOD1 is being synthesized in the central nervous system of the subject;
(c) means for obtaining a biological sample at regular time intervals from the subject, the biological sample comprising a labeled SOD1 fraction and an unlabeled SOD1 fraction; and
(d) instructions for detecting and determining the ratio of labeled to unlabeled SOD1 over time so that a metabolic index may be calculated, whereby the metabolic index may be compared to the metabolic index of a normal, healthy individual or compared to a metabolic index from the same subject generated at an earlier time.

14. (canceled)

15. (canceled)

16. The kit of claim 13, wherein the labeled amino acid has a radioactive or a non-radioactive atom.

17. The kit of claim 16, wherein the non-radioactive atom is selected from the group consisting of ²H, ¹³C, ¹⁵N, ¹⁷O, ¹⁸O, ³³S, ³⁴S, and ³⁶S.

18. The kit of claim 17, wherein the amino acid is leucine and the non-radioactive atom is ¹³C.

19. The kit of claim 13, wherein the labeled amino acid is administered to the subject intravenously.

20. The kit of claim 13, wherein the biological sample is cerebral spinal fluid.

21. The kit of claim 13, wherein the ratio of labeled SOD1 to unlabeled SOD1 is determined from the amounts of labeled and unlabeled SOD1 detected by mass spectrometry.

22. The kit of claim 13, wherein the metabolic index comprises the fractional synthesis rate (FSR) and the fractional clearance rate (FCR).

23. (canceled)

24. (canceled)
